(19) United States
(12) Patent Application Publication
(10) Pub. No.: US 2020/0390066 A1
(43) Pub. Date: Dec. 17, 2020

(54) REMOVABLE TUBE SYSTEM FOR ASSISTING A MALE ANIMAL TO MASTURBATE

(71) Applicants: Hugh Nguyen, Cypress, CA (US); Bao Chau Le, Cypress, CA (US); Hung Quoc Le, Cypress, CA (US)

(72) Inventors: Hugh Nguyen, Cypress, CA (US); Bao Chau Le, Cypress, CA (US); Hung Quoc Le, Cypress, CA (US)

(21) Appl. No.: 16/440,802

(22) Filed: Jun. 13, 2019

(51) Int. Cl. A01K 21/00; A61F 5/41

(52) U.S. Cl. CPC A01K 21/00; A61H 99/00; A61H 2201/0107; A61F 5/41

(57) ABSTRACT

An apparatus for assisting a male animal to masturbate can include: a tube with an end opening into a cavity, the end having a pair of flat surfaces extending therefrom; a plate with an receptacle extending therefrom, the receptacle corresponding to the tube to receive the same upon insertion, the pair of flat surfaces of the end engaging with a corresponding pair of orifices formed through the plate assembly to reversibly lock the tube against the plate. A female animal mannequin can have the plate installed therein. The tube and the receptacle of the plate can be approximately the same length to accommodate coupling there-between. Further, the pair of flat surfaces can each include a protrusion that removably engages with each orifice to reversibly lock the tube with the plate prior to masturbatory use of the tube by a male animal.

### TECHNICAL FIELD

This disclosure relates to a sex toy for masturbatory use by a male animal; and, more particularly, to a replaceable tube that removably engages with a baseplate that is installed in a female animal mannequin suitable for mounting by a sexually aroused male animal.

### BACKGROUND

Conventional sex toys are configured to accommodate the male or female genitals of humans and thus may not be well-suited for animal use due to significant differences in anatomy between humans and animals. Artificial female vagina replacement devices replicate the passageway of a human vagina, often featuring sizing, padding, lubrication, and pleasurable friction-generating surfaces suitable for the insertion and repetitive thrusting of an erect human male penis intended to mimic actual sexual intercourse. Such devices often may be hand-held, allowing for convenience and portability.

These sex toys, however, have failed to provide solutions geared for animals, e.g., related to animal breeding or sexually-motivated aggression control purposes. Sex toys developed specifically for animal use, for instance, may provide a sanitary and humane means for male animals to achieve orgasm and ejaculate semen, thus resulting in a reduction of unwanted pregnancies, excitement and aggression in such male animals.

Solutions have been long sought but prior developments have not taught or suggested any complete solutions, and solutions to these problems have long eluded those skilled in the art. Thus, there remains a considerable need for devices, systems, and methods for use thereof that can assist with the masturbation of a sexually aroused male animal.

### SUMMARY

An apparatus for assisting a male animal to masturbate can include: a tube with an end opening into a cavity, the end having a pair of flat surfaces extending therefrom; and a plate with a receptacle extending therefrom, the receptacle corresponding to the tube to receive the same upon insertion, the pair of flat surfaces of the end engaging with a pair of openings formed through the plate to reversibly lock the tube against the plate. In an embodiment, the plate is installed in a female animal mannequin. The tube and the receptacle can approximately the same length for coupling there-between. Each flat surface can have a protrusion extending therefrom for reversible engagement with each opening such that each protrusion slides along a passage extending from each opening upon rotation of the tube relative to the plate.

A system for masturbation by a male dog can include: a female dog doll with an orifice formed therein, the orifice resembling a vagina of a female dog and having a tube extending towards a head of the female dog doll; a baseplate installed in a rear region of the female dog doll, the baseplate having holes surrounding the orifice, each hole of the holes having a conduit extending therefrom; and a container insertable into the orifice and the tube, the container having fasteners extending therefrom that engage with the conduits of the holes to removably couple the container to the baseplate. In an embodiment, the container is removable from the orifice of the baseplate for cleaning of semen ejaculated therein. The container can be machine-washable. The container can be available in various colors, designs and/or materials. Disengagement of the fasteners from the conduits can remove the container from the baseplate.

A method for assisting a male animal to masturbate can include the steps of: providing a female animal mannequin to the male animal, the female animal mannequin having an orifice opening inwardly therein; engaging a tubular insert with the orifice for receiving the insertion of an erect penis of the male animal to simulate sexual intercourse with the female animal mannequin; positioning the orifice of the female animal mannequin to provide accessibility to the male animal; and cleaning the tubular insert after ejaculation of semen there-into by the male animal. In an embodiment, the tubular insert can be replaced with an unused tubular insert prior to repeat masturbatory usage of the female animal mannequin.

Other contemplated embodiments can include objects, features, aspects, and advantages in addition to or in place of those mentioned above. These objects, features, aspects, and advantages of the embodiments will become more apparent from the following detailed description, along with the accompanying drawings.

### BRIEF DESCRIPTION OF THE FIGURES

The removable tube for assisting a male animal to masturbate is illustrated in the figures of the accompanying drawings which are meant to provide examples only and are thus not limiting, in which like reference numerals are intended to refer to like components, and in which:

FIG. 1 is a representative diagram of a female dog mannequin with the removable tube system installed therein for assisting a male dog to masturbate.

FIG. 2 is a cut-away illustration of the reproductive organs of a female dog.

FIG. 3 is a cut-away illustration of the reproductive organs of a male dog.

FIG. 4 is photograph of two dogs mating.

FIGS. 5A-D are views of the removable tube for assisting a male animal to masturbate.

FIG. 6 is a representative drawing of the female dog mannequin shown in FIG. 1 with the removable tube shown in FIGS. 5A-D installed therein.

FIG. 7. is a flow chart for a method of operation of a system with a removable tube for assisting with the masturbation of a male animal.

### DETAILED DESCRIPTION

### General Techniques

In the following description, reference is made to the accompanying drawings that form a part hereof, and in which are shown by way of illustration, various embodiments relating to a removable tube system for assisting a male animal to masturbate that may be practiced. It is to be understood that other embodiments may be utilized and structural changes may be made without departing from the scope of the removable tube apparatus and/or system.

When features, aspects, or embodiments of a method of use of the removable tube are described in terms of steps of a process, an operation, a control flow, or a flow chart, it is to be understood that the steps can be combined, performed in a different order, deleted, or include additional steps without departing from the removable tube system for assisting a male animal to masturbate as described herein.

The removable tube system is described in sufficient detail to enable those skilled in the art to make and use the same and provides numerous specific details to give a thorough understanding thereof; however, it will be apparent that the removable tube system may be practiced without these specific details.

To avoid obscuring the removable tube system some well-known system configurations are not disclosed in detail. Likewise, the drawings showing embodiments of the system are semi-diagrammatic and not to scale and, particularly, some of the dimensions are for the clarity of presentation and are shown greatly exaggerated in the drawing FIGS. Generally, the removable tube system can be operated in any orientation and/or configuration.

Various aspects of the present disclosure are directed to a system for assisting with a sexually excited or aroused male animal to masturbate. More specifically, and as described in greater detail below, various aspects of the present disclosure are directed to a tube that can be inserted into a baseplate assembly, which collectively can be installed in a female animal mannequin for masturbatory use by the male animal. The tube can be detached from the baseplate assembly after ejaculation of the male animal there-into for cleaning prior to re-use or re-application of the tube.

### Introduction

Studies have monitored the social behavior of various animals as related to a number of factors including aggression due to lack of regular sexual outlet or release for domesticated animals (as may otherwise be available in the wild). [Source: “The Social Biology of the Olympic Marmot”; David Philip Barash; Animal Behavior Monographs; Vol. 6, Part 3, pp. 171-245]. For example, sexual aggression in male dogs may be seen in their efforts to mount humans as well as other dogs for self-pleasuring (e.g., masturbation) and release (e.g., ejaculation of semen). [Source: https://www.doglistener.co.uk/aggression/types.shtml; Retrieved on May 6, 2019]. Moreover, the mounting of dogs on other dogs may be related to rank in and other complex group-related social behavior. Neutering (e.g., castration) may reduce such behavior but introduce other, undesirable, side effects associated with the reduced production of male hormones, such as lowered testosterone and the reduction in other vital hormones such as serotonin, resulting in mood changes and the possible deterioration of the overall quality of life of the neutered male dog.

Simply allowing male dog to freely mate with other female dogs, as would occur in nature, may alleviate such concerns, but is not always feasible due to monetary, geographic, social or other constraints. And, male dogs do not necessarily mount female dogs, other male dogs, or even inanimate objects such as sofas, only out of sexual desire or instinct—rather, certain male dogs may perceive mounting as a form of demonstrating social dominance in a group hierarchy or stress relief. [Source: https://www.whole-dog-journal.com/issues/7_3/features/Mounting-and-Dominance_5615-1.html; Retrieved on 05-06-19].

Although dogs may be trained to repress such undesirable behavior (e.g. “humping”), such training may not address the core cause of the issue—that the male dog has not adequately been permitted to release built-up sexual tension or frustration by masturbating to reach an orgasm and the pleasurable ejaculation of semen. Moreover, adult dogs that have not been trained as puppies may relapse into undesirable masturbatory behaviors, and also demonstrate aggression when prevented from masturbating as desired.

Such a situation often leaves dog owners and enthusiasts in a quandary—introducing an artificial limitation on natural male dog sexuality may be felt as cruel, while training only limits natural urges without providing a safe and suitable sexual outlet. In a human context, sex toys and devices fill this void by providing an alternative to actual sexual intercourse by artificially replicating aspects of coitus permitting users to achieve climax or orgasm as desired, e.g., male users ejaculating semen into the toys or devices. The following issued patents, incorporated herein by reference in their respective entireties, discuss sex toys and devices:

U.S. Pat. No. 10,045,906 entitled “System and Methods for Haptic Stimulation” relates to a system for sexual stimulation having a first sexual stimulation device with a first sensor and a first actuator, and a second sexual stimulation device having a second sensor and a second actuator. The first sexual stimulation device communicates information from the first sensor to actuate the second actuator of the second sexual stimulation device. The second sexual stimulation device is configured to communicate information from the second sensor to actuate the first actuator of the first sexual stimulation device.

U.S. Pat. No. 5,571,118 entitled “Apparatus for Stimulating Penile, Scrotal, Anal, Vaginal, and Clitoral Tissue” relates to male and female electrodes for stimulating penile, scrotal, anal vaginal and clitoral tissue. The electrodes are formed from elastomeric material. Electrical stimulation to such areas is intended to control incontinence, to induce penile erection where impotence occurs, or to induce excitation and orgasm.

U.S. Pat. No. 7,608,037 entitled “Remotely Located Pleasure Devices” relates to a sexual preference transfer function that combines the masturbatory action of a first person with the expressed or non-expressed sexual preferences of a second person. A sexual preference transfer function is utilized to improve the control or influence of a sexual pleasure device in contact with a second person based on autonomic and/or manually collected data.

U.S. Pat. No. 8,308,631 entitled “Electronic Variable Stroke Devices and System for Remote Control and Interactive Play” relates to an electronic variable stroke device having a base portion containing a motor-driven screw shaft. An upper portion extends from the base portion and has the screw shaft extending longitudinally therein. A traveler is engaged with the screw shaft to drive it in reciprocating longitudinal motion. The traveler has an annular shape with an aperture therethrough which is driven in reciprocating longitudinal motions for use as a male sex toy. A network connection unit may be provided to connect the user's device to an external service provider on a network for conducting interactive sessions remotely with another user or users. The device may be adapted as a male toy that can exchange control signals with another user operating a female toy.

As can be seen from the issued patents discussed above, such solutions appear limited or not available entirely in animal contexts, as innovation in the area has been typically limited to only animal play, feeding, and behavioral adjustment or retention with no specific provision for sexual outlet or gratification of animals as possible through masturbation. Human sex toys, devices, and methods, such as those presented above, fail to adequately address or meet the unique masturbatory needs of animals, given the significant differences in the general body structure and reproductive anatomies of humans and animals. Nevertheless, although animal-related toys and products do exist, they often fail to specifically address the sexual needs of animals, and may not provide a convenient configuration or orientation allowing for animals to mount such toys to masturbate to achieve orgasm. The following issued patents, incorporated herein by reference in their respective entireties, discuss pet or animal toys or devices:

Chinese Patent No. CN 202907602U entitled “Pipe-Shaped Pet Toy” relates to a pet toy, particularly to a pipe-shaped pet toy that addresses an unmet need regarding substantially autonomous (e.g., without human interaction or involvement), pet engagement and/or entertainment. The pipe-shaped pet toy comprises a pipe-shaped body, at least a food loading piece, a control switch, a connecting rod and a buffering sliding slope. A food leaking opening is formed at the bottom of the pipe-shaped body. Every food loading piece has an object loading piece that is arranged inside the pipe-shaped body and matched with the inner wall of the pipe-shaped body. A control switch is used for controlling relative positions of object loading pieces and the pipe-shaped body. A connecting rod penetrates through a pipe wall, where one end of the connecting rod is connected with the object loading pieces, and the other end of the connecting rod is connected with the control switch. The pipe-shaped pet toy has the advantages of providing a connection between pet actions and automatic drop of food and is capable of enabling pets to engage in self-play in an unaccompanied condition.

French Patent No. FR2794358 A1 entitled “Female Dog Sex Prosthesis” relates to a sexual toy for a male dog and has three apertures for inserting fixing straps, a genital orifice coated with an odoriferous liquid, and receiving bag for collecting sperm. The toy can be attached to any support, but preferably simulating shape of dog. Genital orifice is coated with special odoriferous liquid (pheromone) so as to attract a male dog.

Korean Patent No. KR200401337 Y1 entitled “Toy Having [a] Multi-Function” relates to multifunctional toys for pet dogs, cats, birds depending on the particular model selected, and permits for a user to manipulate the appearance of the animal. Magnetic sensors are in contact sensors of opposing polarity in each toy. A speech recognition unit recognizes a user's voice. A predetermined portion of a front light emitting part emits light. An audio output unit provides a predetermined output sound. The toy of the subject invention can express a variety of operations by recognizing a user operation of various types to maximize a user's interest and curiosity and reduce tire.

Mounting, thrusting (e.g., humping) and masturbation are normal behaviors shown by many dogs, who masturbate in various ways, including: mounting and thrusting against other animals, people, and objects, such as blankets, dog beds and toys. [Source: https://www.aspca.org/pet-care/dog-care/common-dog-behavior-issues/mounting-and-masturbation; Retrieved on May 6, 2019]. Moreover, “[i]ntact (not neutered) males will often masturbate if prevented from approaching a female in heat.” [Source: https://www.aspca.org/pet-care/dog-care/common-dog-behavior-issues/mounting-and-masturbation; Retrieved on May 6, 2019]. Consequently, such intact male dogs need a reliable solution permitting for the alleviation of pent-up sexual desire or tension.

### Embodiments

### Definitions

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,”, “male,” “female”, and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The terms “couple,” “coupled,” “couples,” “coupling,” and the like should be broadly understood and refer to connecting two or more elements mechanically and/or otherwise. Coupling may be for any length of time, e.g., permanent or semi-permanent or only for an instant. “The absence of” the word “removably,” “removable,” and the like near the word “coupled,” and the like does not mean that the coupling, etc. in question is or is not removable.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The following terms and phrases, unless otherwise indicated, shall be understood to have the following meanings.

The term “tube” refers to a substantially cylindrical body made from a rigid material, such as metal, glass, and/or rubber and has a hollow interior. The tube is insertable into a corresponding annular receptacle in a female plate assembly for reversibly locking therewith prior to usage by a male animal for masturbation. The tube is suitable here for application with a lubricant, padding and/or coating applied on internal surfaces to receive the erect penis of a sexually aroused male animal, such as a dog.

The term “end” refers to the last part or extremity, lengthwise of anything that is longer than it is wide or broad. Here, “end” may be used for the tube, referring to an opening to receive the erect penis of a sexually excited animal.

The term “cavity” refers to any hollow place. Here, “cavity” may be used to refer a hollow region within the tube extending from an open end thereof

The term “flat surface” refers to a projecting rim, collar, or ring on a shaft, pipe, tube, machine housing, etc., cast or formed to give additional strength, stiffness, supporting area, or to provide a place for the attachment of other objects. Here, “flat surface” may be used to refer to a pair of opposed surfaces extending outwardly from the tube to assist with attachment to other surfaces.

The term “female plate assembly” refers to a flat plate with a tube extending perpendicularly therefrom, the flat plate being configured with holes and/or openings to receive corresponding protrusions extending from the flat surfaces of the tube for coupling therewith.

The term “annular receptacle” or “receptacle” refers to the tube extending perpendicularly from the female plate assembly; the annular receptacle being sized to receive, combine and/or otherwise engage with the tube upon insertion therein.

The term “engaging” refers to removably and/or fixedly securing on object with another; as understood in the disclosure herein, “engaging” also encompasses any and all intermediary objects positioned between at least two or more principal objects sought for combination and/or attachment.

The term “female animal mannequin” refers to a styled three-dimensional (“3D”) representation of the animal form; the “female animal mannequin” may be substantially formed from and/or with soft-touch materials and surfaces permitting for convenient and pleasurable mounting and penetration by a sexually aroused male animal.

The term “protrusion” refers to something that projects.

The term “adjustable” refers to the capability of being adjusted, e.g., in direction, size, orientation, configuration, purpose and/or to accommodate a specific use.

The term “masturbation” refers to the stimulation of one's own genitals, often to orgasm and/or otherwise referring to sexual self-gratification. In an animal-related context as discussed herein, “animal masturbation” refers to masturbation when partners may or may not be available for traditional coitus.

The term “baseplate” refers to a flat plate from which the annular tube extends perpendicularly therefrom; the baseplate is further a part of the female plate assembly.

The term “hole” refers to an opening through something, a gap and/or an aperture. Here, holes formed in the baseplate of the female plate assembly receive protrusions extending from the flat surfaces of the tube for removable engagement.

The term “conduit” refers to a passage, e.g., extending outwardly in a linear direction from the hole formed in the baseplate; the conduit may receive the protrusion, which may slide further into the conduit to fixedly attach the tube to the female plate assembly.

The term “sheath” refers to a case or covering; here, the “sheath” may be used interchangeably to describe the tube.

### Generic Description of the Disclosed Embodiments

Sexual intercourse is a pleasurable and rewarding life experience for many adult humans, providing for a release of pent-up sexual tension or desire. For interested persons, sex and/or sexual toys may be used to either supplement sex acts with other persons or fill the need to engage in a sex act with another human altogether, effectively replicating the reproductive organs of another party dependent on need, e.g., devices replicating vaginas for masturbatory use heterosexual males, and so on and so forth. As generally understood, a sex toy is an object or device that is primarily used to facilitate human sexual pleasure, inclusive of such devices and objects such as a dildo, vibrator, or artificial vaginas. Regular cleaning of such toys can be important for sexual health and sex toy safety to prevent against the potential of bacterial infection, the transmission of sexually-transmitted-infections, or pregnancy (if residual sperm is present on the toy).

Like humans, many animals also engage in mating or reproductively motivated behavior. For instance, females often select males for mating based on dominant characteristics such as demonstrated strength and the ability to protect of the males to protect themselves and others. Dominant males who prevail in combative encounters often have the opportunity to mate with larger numbers of females, thus increasing the likelihood of passing on their genetic material to any resultant offspring. [Source: “The sexual code: the social behavior of animals and men”; Wickler, W.; Lorenz, K., Kacher, H.; 1974, Weidenfeld and Nicolson].

Moreover, although historically it may have been believed that only humans and a small number of animal species participated in performing sexual acts for purposes other than reproduction, recent findings have indicated that animals do masturbate, as humans do, for self-pleasure and may even use objects as tools for assistance in such endeavors. [Source: “Object Play and Tool Use: Developmental and Evolutionary Perspectives”; Bjorklung, D. F.; Gardiner, A. K.; 2011; in The Oxford Handbook of the Development of Play; Pellegrini, A. D. (ed.); Oxford University Press; p. 153]. Also, sexual behavior may be tied to the establishment and maintenance of complex social bods across a population, which may support the success of the population in non-reproductive ways. To summarize, both reproductive and non-reproductive behaviors can be related to expressions of dominance over another animal or survival within a stressful situation.

More particularly, as many pet owners and enthusiasts may be aware, domesticated dogs and cats may be fond of masturbation. In that regard, “mounting, thrusting (humping) and masturbation are normal behaviors exhibited by most dogs,” according to the American Society for the Prevention of Cruelty to Animals (ASPCA). [Source: https://www.livescience.com/12944-animals-evolved-masturbate.html; Retrieved on: May 9, 2019]. For instance, dogs enjoy thrusting and rubbing their genitalia against objects, people and animals, and also enjoy licking themselves. Cats, similarly, also enjoy self-fellation.

Devices currently available fail to address animal masturbatory needs, failing to effectively bridge the gap between sex toys suitable for human use, and traditional animal toys for play. As a result, animals are forced to resort to traditional masturbatory techniques as so described, which may be undesirable for their owners in social settings, where such “humping” may be distractive and interfere with, for example normal family time together. Accordingly, the disclosed inventive apparatus, system and/or method of use thereof seeks to address such unmet needs by essentially replicating the vagina and/or vaginal passage of a female animal, such as a domesticated dog, suitable for entry and rhythmic repetitive movement of an erect male penis, e.g., similar to actual sexual intercourse.

### System Structure

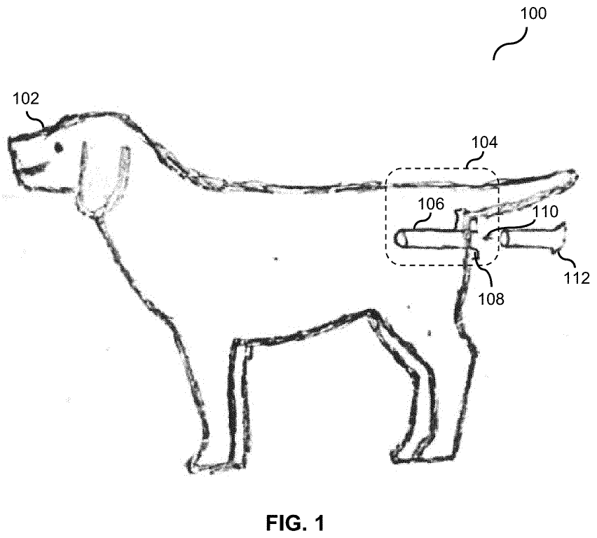

Referring now to FIG. 1, therein is shown a diagram of a female animal doll or mannequin 100, which can include elements of a head 102, rear region 104, orifice 108 opening to an artificial vagina or vaginal passageway 106. Representative tube 112 may be inserted in a direction 110 into the vaginal passageway 106 via the orifice 108 for reversible engagement with the vaginal passageway 106 prior to mounting by a sexually excited male animal for masturbatory use, e.g., of the same or of a different species.

Regarding usage, the female animal mannequin 100 may be positioned by a caretaker, e.g., a dog owner, in front of a male animal, e.g., a dog, that is becoming sexually aroused, or is already in such a condition. The excited male animal, experiencing an erection (of his penis), will process to insert his erect penis into the tube 112 to begin rhythmic back-and-forth movement for the purposes of self-pleasure, much as in the case of actual sexual intercourse between two animals, or during masturbation. The male animal can mount the rear section 104 to penetrate the tube 112 that is already installed in a locking position in the vaginal passageway 106 via the orifice 108.

One skilled in the art will appreciate that the representation of a dog shown for the female animal mannequin 100 is for example purposes only, and that other suitable configurations are possible for other animals, such as those geared for cats, horses, cows/bulls, walruses and/or the like. Moreover, it is contemplated that the positionings of each the head 102 and the rear region 104 are approximate and may be dependent on the species of animal represented by the female animal mannequin 100. For instance, the anatomic representations of the head 102 and the rear region 104 may be different for a cat or horse than that of a dog. Still further, placement of the vaginal passageway 106 within the female animal mannequin 100 may be preconditioned upon the preferences, or expected preferences, of the corresponding male of the species intended to use the female animal mannequin 100 for masturbatory purposes and/or sexual release. Accordingly, such placement may also depend at least in part on the sub-species or breed of animal as well.

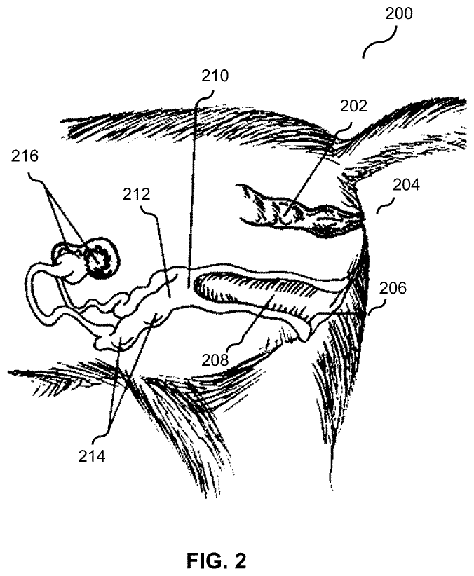

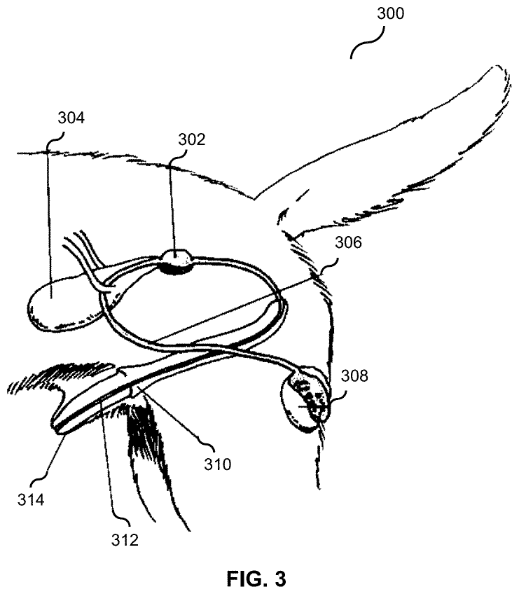

FIGS. 2 and 3 are illustrations of the female and male reproductive systems of dogs, respectively. The female animal mannequin 100 seeks to generally replicate the anatomy of the female reproductive system, as shown in FIG. 2, of an animal for the masturbatory use of a male animal, e.g., often of the same species, but potentially of a similar or different species as well. Elements shown and discussed with reference to FIG. 1 are also representative, where such elements may include fewer or additional elements not explicitly shown and discussed but also potentially relevant regarding the creation of an artificial replica of the female reproductive system of an animal.

Referring now to FIG. 2, a female reproductive system 200 of an animal, e.g. a dog, is shown with the following organs, areas, and/or anatomical features: an anus 204, a colon 202, a vagina 206, a vulva 208, a cervix 210, a uterine horn 212, developing embryos 214, an ovaries 216. The anus 204 provides an outlet for waste to be excreted from the colon 202, which connects to the intestine and general digestive system (not shown in FIG. 2).

In a canine, as may be the case for other species as well, the vagina 206 refers to the general region into which an erect penis may be inserted for coitus, and may be effectively replicated or reproduced by the vaginal passageway 106 of the female animal mannequin 100 shown in FIG. 1. It is not contemplated that the other functional organs of the female animal reproductive system inclusive of the developing embryos 214 and ovaries 216 will need to be replicated in the female animal mannequin 100, given that such organs may not be needed to provide a complete masturbatory solution for a sexually aroused male animal, but rather serve in actual reproduction. However, in an embodiment, the uterine horn 212 may be fabricated as a part of or an extension of the vaginal passageway 106 to enhance male animal pleasure, if necessary or appropriate in certain configurations, upon entrance of the erect penis of the excited male animal.

Referring now to FIG. 3, a male reproductive system 300 of an animal, e.g. a dog, is shown for explanatory purposes related to usage of the female animal mannequin 100 shown in FIG. 1, and may include the following organs and/or anatomical features: a penis 314, a urethra 312 extending there-through, a bulbus glandis 310, testicles 308, a vas deferens 306 extending from the testicles 308, the vas deferens 306 also connecting with a prostrate 302, and a bladder 304. One skilled in the art will appreciate that the discussion herein is representative, and that other configurations of genitalia may exist dependent on, for example, the species of male animal discussed. Specifically, upon (for example) receipt of visual, auditory, or haptic stimulation that is sexually attractive in nature, a male animal may become aroused resulting in increased blood flow to the penis 314 resulting in an erection thereof. Other forms of excitement resulting in an erection of the penis 314 may also exist, including efforts to exert dominative behavior in groups, or also resultant of contact and/or friction against another surface.

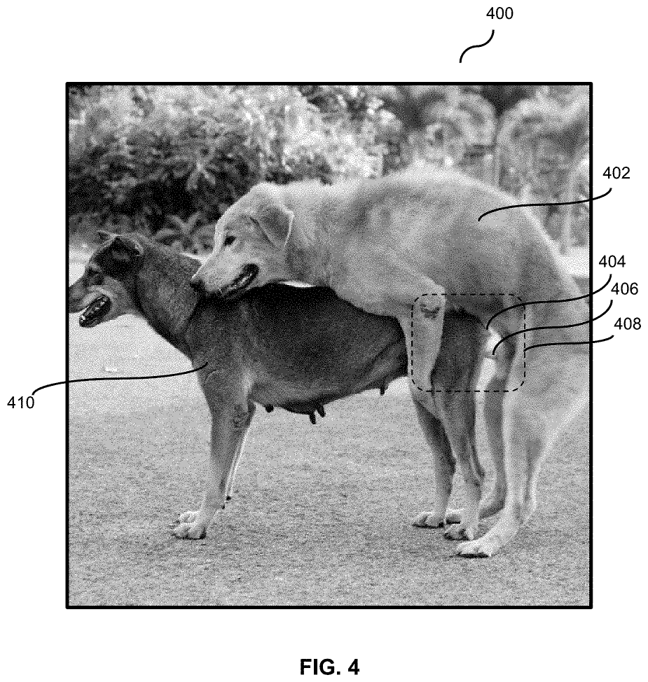

Stimulation of the genitalia of a male animal, e.g., dog, may result in efforts of the dog to mount a female dog as generally depicted in photograph 400 shown in FIG. 4, which provides an example of a possible position of the female animal mannequin 100 for masturbatory use by a sexually aroused male animal. Here, a male dog 402 is sexually aroused and thus achieves an erection 406 to penetrate a vagina 404 of a female dog 410 within a general penetration region 408. In an embodiment, the female animal mannequin 100, shown in FIG. 1, may be put in place of the female dog 410, with the rear region 104 of the female dog mannequin 100 positioned generally adjacent to the erection 406 of the male dog 402. Thus, the male dog 402 may insert his erection into the tube 112, e.g., positioned within and/or coupled with vaginal passageway 106. In an embodiment, the tube 112 may be prepared with soft-touch surfaces lining an interior circumference of the tube 112, and also feature lubrication and/or stimulating gels and/or creams to enhance the pleasure and general experience of the male dog 402 as he rhythmically thrusts his erection 406 in, out, and throughout the tube 112 installed in the vaginal passageway 106 to achieve orgasm and to ejaculate semen into the tube 112.

Accordingly, the real act of coitus between a male dog, or animal more generally, is effectively replicated by the female animal mannequin 100, permitting for the convenient and desirable release of pent-up or built-up sexual tension or energy of the male dog 402 upon ejaculation into the tube 112 installed in the female dog mannequin 100. The sexual position of the male dog 402 and female dog 410 is provided as a representative example in photograph 400. One skilled in the art will appreciate that other possible positions for sexual intercourse may exist without departing from the scope and spirit of the disclosed embodiments. Thus, the female animal mannequin 100 may be placed or positioned in an orientation different from that shown in photograph 400; e.g., in a position other than where the female dog 410 is currently shown. To accommodate such differences in positions, the various features and/or elements of the female animal mannequin 100, such as the vaginal passage 106, may be repositioned or reconfigured within the female animal mannequin 100 as needed to accommodate successful penetration by a sexually excited male animal.

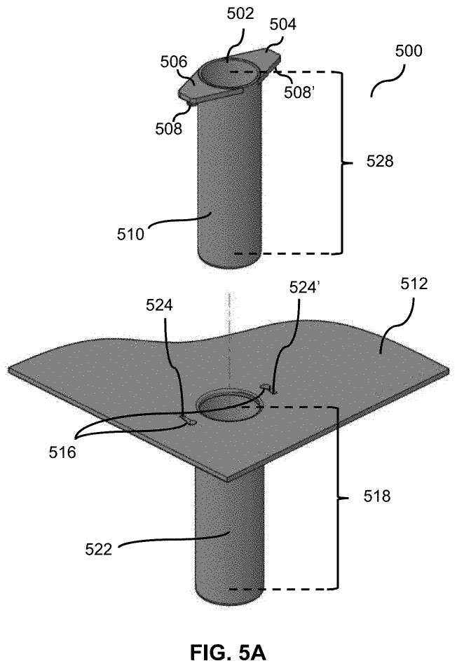

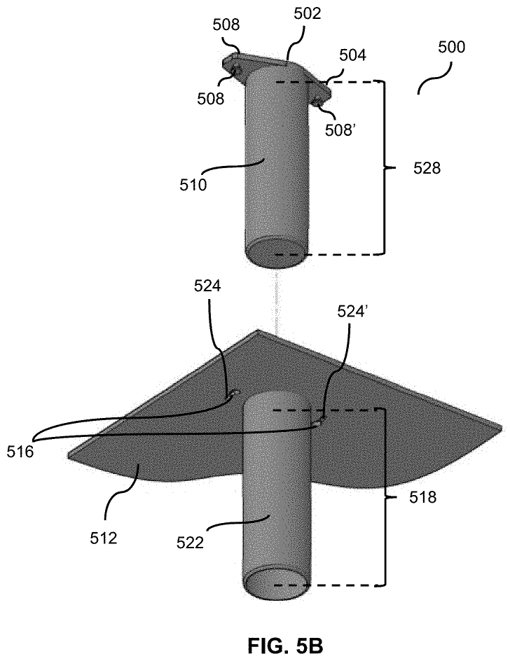

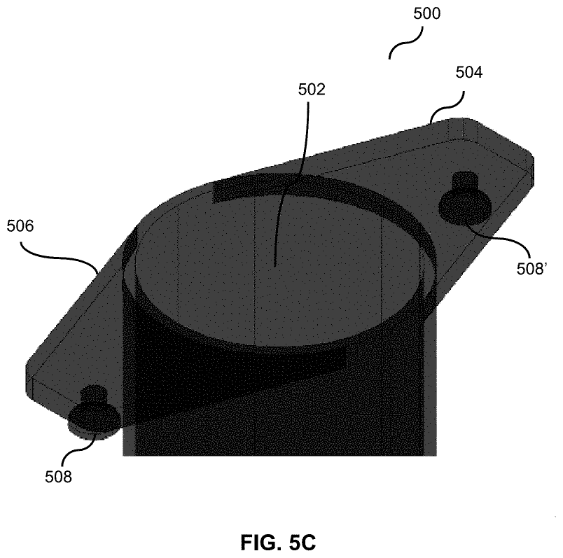

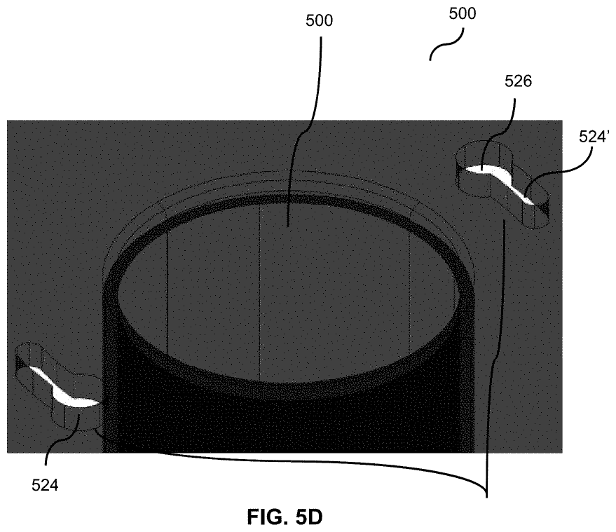

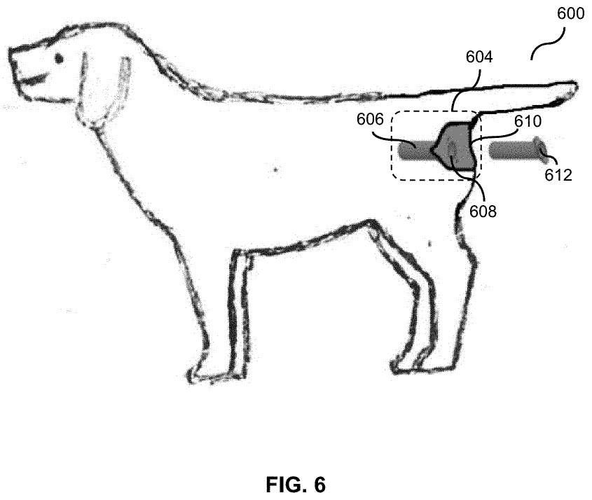

Referring collectively now to FIGS. 5A through 5D, therein is shown a removable tube system 500 for masturbatory use of a male animal. In an embodiment, the removable tube system 500 may be installed in a female animal mannequin 600 so prepared as shown in FIG. 6. The female animal mannequin 600, as shown in FIG. 6, may be substantially similar to the female animal mannequin 100 shown in FIG. 1, but not necessarily so, and thus can include variations specific to the accommodation and/or usage of the removable tube system 500.

Referring specifically to FIG. 5A, the removable tube system 500 includes, in an embodiment, a tube 510 and a baseplate assembly 530 corresponding therewith. The tube 510 that can be inserted in a direction 534 into an opening 536 of an annular receptacle 522, which extends perpendicularly from the baseplate assembly 530 in the direction 534.In an embodiment, the direction 534 is at least substantially parallel to a vertical axis 532 extending from the tube 510 toward the baseplate assembly 530 and the annular receptacle 522 extending therefrom.

In an embodiment, the tube 510 may be referred to as a “male tubular insert” referring to the capability of the tube 510 to penetrate into the baseplate assembly 530 to removably or reversibly interlock therewith. Such interlocking may be accomplished by aligning a first and second flat surface 506, 504, respectively, both extending outwardly from the tube 510, with openings 516. The openings 516 are formed in and extend through a baseplate 512 of the baseplate assembly 530.A first protrusion 508 extends outwardly from the first flat surface 506 and a second protrusion 508′ extends outwardly from the second flat surface 504. In an embodiment, the shape, size, position and/or formation of the first and second protrusions 508 and 508′ may be substantially similar to one-another, and be shaped to fit and/or otherwise engage with the openings 516 to removably attach the tube 510 to the annular receptacle 522.

The mechanism through which the tube 510 engages with and removes from the annular receptacle 522 by reversible engagement of the protrusions 508, 508′ with their corresponding openings 516 in the baseplate 512 is described as follows: (1) the tube 510 is at least substantially aligned with the baseplate assembly 530 along the vertical axis 532; (2) the tube is inserted, e.g., moved in the direction 534, into the annular receptacle 522 until the protrusions 508, 508′, insert into the openings 516; and, (3) rotating the tube 510 in a direction “A” as shown in FIG. 5A such that the protrusion 508 slides into a first conduit 524 (extending from one of the openings 516), and that the protrusion 508′ slides into a second conduit 524′ (extending from another one of the openings 516). In an embodiment, the direction of extension of the conduits 524 and 524′ are substantially opposite to one-another, permitting for the locking or engagement of the protrusions 508 and 508′ with the openings 516 to secure the tube 510 against the baseplate 512 of the baseplate assembly 530. In an embodiment, rotation of the tube 510 in the direction “A” can result in sealing the flat surfaces 506 and 504 against the baseplate 512, providing for a secure and reliable engagement of the tube 510 to the annular receptacle 522 as may be desirable to receive the erect penis of a male animal using the removable tube system 500 to masturbate.

The baseplate assembly 530 may be referred to as a “female baseplate assembly” in view of its capabilities to receive the corresponding “male tubular insert”, another term for the tube 510 as discussed earlier. Such mating of the “male tubular insert” with the “female baseplate assembly” combines the tube 510 with the baseplate assembly 530 for masturbatory usage by a sexually aroused male animal.

After the sexually excited male animal repeatedly thrusts his erect penis in and out of the tube 510 by first entering into the tube 510 by an opening, a hole, or an end, 502 thereof, the male animal may ejaculate semen into the tube 510 upon reaching an orgasm. In an embodiment, the interior-facing circumferential surface and/or surfaces of the tube 510 may include padding, lubrication, gels, and/or fragrances, any one or more designed or intended to enhance the pleasure and/or the safety of the male animal while masturbating with or into the tube 510. The tube 510 can be, in an embodiment, having a height 528 ranging from at least 2 to a maximum of 9 inches and a width of 1 to 6 inches to accommodate a variety of sizes, shapes, and orientations of erect animal penises. The height 528 of the tube 510 can substantially match a corresponding height 518 of the annular receptacle 522 such that the tube 510 can conveniently enter into and remain locked in position within the annular receptacle 522 as so described.

Subsequent to usage and ejaculation by a male animal, the tube 510 may be dis-engaged from the baseplate 512 of the baseplate assembly 530 for removal, cleaning, and potential replacement with a new, clean, and/or different tube 510 for later re-use by the same or another sexually aroused male animal. Procedures for removal of the tube 510 from the baseplate assembly 530 are presented as follows: (1) rotation of the tube in a direction “B” substantially opposite to the direction “A” discussed earlier dis-engages the protrusions 508 and 508′ from their corresponding conduits 524 and 524′, effectively sliding the protrusions 508 and 508′ into larger first and second circular openings 526, 526′, respectively, as shown in FIG. 5D, of openings 516; (2) such sliding permits for the lifting of the tube 510 away from the baseplate 512 opposite to the direction 534; (3) the used tube 510, e.g., having ejaculated animal semen therein, can then be cleaned to be re-inserted into the baseplate assembly 530 as described above prior to subsequent use by another or the same male animal, or the tube 510 may be replaced by a fresh and/or new tube 510.

One skilled in the art will appreciate the configuration of the removable tube system 500 is an example and that other suitable configurations may exist without departing from the scope and spirit of the disclosed embodiments. For instance, the angle of attachment of the baseplate 512 and the annular receptacle 522 extending therefrom may differ from that shown in FIG. 5A, and the lengths 528 and 518 of the tube 510 and annular receptacle 522, respectively, may also differ from that shown in FIG. 5A. Further, the baseplate 512 need not necessarily be flat and may be formed in any curvature or configuration suitable for attachment, combination or installation with the female animal mannequins 600 or 100 shown in FIGSs. 6 and 1, respectively.

Referring specifically to FIG. 5B, the removable tube system 500 is shown in an elevation or perspective different from that shown in FIG. 5A for illustrative purposes. Like reference numerals refer to like elements and/or features in both FIGS. 5B and 5A, such that a redundant description of the same will be omitted herein for brevity.

Openings 516 and conduits 524 and 524′ extending therefrom are shown as clearly extending through the baseplate 512 to receive and/or engage with protrusions 508 and 508′ extending from flat surfaces 506 and 504, respectively. In an embodiment, the protrusions 508 and 508′ have first and second stop portions 538, 538′, respectively, as shown in FIG. 5C, which can be made of a substantially rigid, yet pliable, material (such as hard rubber), suitable for compression to accommodate insertion through the circular portions of the openings 516. This allows for the expanded head section of each protrusion 508 and 508′ to compress against an underside of the baseplate 512 to hold the flat surfaces 506 and 504 against a top side of the baseplate 512. Such a fastening of the first and second stop portions 538, 538′of the protrusions 508 and 508′ against the underside of the baseplate 512 permits for secure and reversible locking of the tube 510 against and/or with the baseplate assembly 530.

In an embodiment, the annular receptacle 522 and the tube 510 may each have a closed end such that the closed end of each compresses against one-another allowing for a secure fit of the tube 510 into the annular receptacle 522. The tube 510 may have a compartment (not shown in the FIGS.) for collecting and retaining semen ejaculated by a male animal upon completion of masturbatory usage of the removable tube system 500.

Other example configurations may exist other than the elevations and/or perspectives shown in FIGS. 5A and 5B without departing from the scope and spirit of the disclosed embodiments. For instance, the angle of insertion of the tube 510 into the baseplate assembly 530 may differ slightly or significantly from that shown by the direction 534. Also, the lengths of the flat surfaces 506 and 504 are shown for example purposes only and may be shorter or longer in practice.

FIGS. 5C and 5D are close-up views of the flat surfaces 506, 504 and of the opening or the end 502 of the tube 510 and of the removable tube system 500 more generally. As shown, both the first and second protrusions 508 and 508′, respectively, extend lengthwise in a direction substantially perpendicular to the flat surfaces 506, 504. In an embodiment, the first protrusion features a first portion 540 that attaches to and extends from the first flat surface 506. Likewise, the second protrusion 508′ features a second portion 540′ that attaches to and extends from the second flat surface 504. One skilled in the art will appreciate that other shapes or forms of the first and second portions 510 and 510′ may exist without departing from the scope and spirit of the disclosed embodiments. In the embodiment shown in FIG. 5C, the first and second portions 510 and 510′ are shown as being cylindrical as an example. The first portion 540 extends from the first flat surface 506 to couple with a first stop portion 538. In an embodiment, the first stop portion 538 can be substantially semi-hemispherical and have a hollowed-out interior, e.g., for the purposes of weight reduction. The first stop portion 538 of the first protrusion 508 can enter into a first circular opening 526 (shown in FIG. 5D) of the openings 516 and slide into the first conduit 524 to secure the second flat surface 506 against the baseplate 512. Likewise, the second portion 540′ extends from the first flat surface 504 to couple with a second stop portion 538′. In an embodiment, the second stop portion 538′ can also be substantially semi-hemispherical and feature a hollowed-out interior. The second stop portion 538′ can enter into a second circular opening 526′ (shown in FIG. 5D) of the openings 516 and slide into the second conduit 524′ to secure the second flat surface 504 against the baseplate 512.

FIG. 5D shows a close-up view of an area of the baseplate 512 specific to receiving insertion of the tube 510 into the opening 536 of the annular receptacle 522. Like numerals refer to like elements as introduced earlier such that a redundant description of the same is omitted herein. For removable engagement of the tube 510 with the baseplate 512 and the baseplate assembly 530 more generally, the protrusions 508, 508′ of the flat surfaces 506, 504, respectively, are inserted into first and second circular openings 526, 526′ of the openings 516. Next, rotation of the tube in direction A as shown in FIG. 5A will slide the first and second stop portions 538, 538′ of the first and second protrusions 508, 508′, respectively, from the first and second circular openings 526, 526′ into their respective conduits 524, 524′ extending outwardly therefrom for engagement therein. The flared and/or enlarged semi-hemispherical geometry of the first and second stop portions 538, 538′ permits for a secure compressive and/or interference-type fit with the conduits 524, 524′ and ends thereof. However, one skilled in the art will appreciate that other potential geometries of the first and second stop portions 538, 538′, their corresponding first and second circular openings 526, 526′, and the conduits 524, 524′ extending therefrom may exist and vary from that shown in FIGS. 5C and 5D without departing from the scope and spirit of the disclosed embodiments.

Disengagement of the tube 510 from the baseplate 512 and the baseplate assembly 530 more generally involves rotation of the tube 510 in the direction B shown in FIG. 5A. Doing so slides the first and second stop portions 538, 538′ of the first and second protrusions 508, 508′, respectively, from their respective conduits 524, 524′ into their corresponding first and second circular openings 526, 526′ of the openings 516. Next, the tube 510 is moved away from the baseplate 512 in a direction opposite to the direction 534, shown in FIGS. 5A and 5B, to remove the tube 510 from the baseplate 512 to permit for, for example, cleaning or replacement of the tube 510.

Referring now to FIG. 6, the female animal mannequin 600 is shown with the removable tube 500 system positioned and installed in a rear region 604 of the female animal mannequin 600. Usage of the female animal mannequin 600 by a sexually aroused male animal may be, in an embodiment, substantially similar to, or identical to, usage of the female animal mannequin 100 shown in FIG. 1. Like reference numerals refer to like elements in FIG. 6, such that a redundant description of the same is omitted.

In an embodiment, the female animal mannequin 600 can be prepared to receive the removable tube system 500 for installation in the rear region 604 for masturbatory usage by a male animal substantially as described for the female animal mannequin 100 shown in FIG. 1. That is, the sexually excited male animal can insert his erect penis into the opening 502 of the tube 510 after attachment thereof to the baseplate assembly 530. The male animal may next use the removable tube system 500 to masturbate to achieve orgasm and ejaculate semen into the removable tube 510, which may then be disengaged from the baseplate assembly 530 for cleaning.

### Method of Working the System

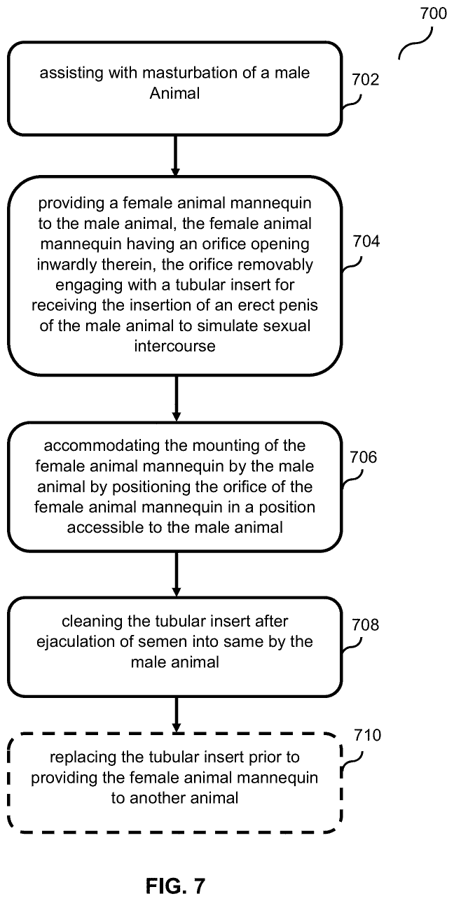

Referring now to FIG. 7, therein is shown a method 700 for assisting with the masturbation of a male animal with, for example, the female animal doll or mannequin 600 shown in FIG. 6 having the removable tube system 500 shown in FIGS. 5A-5D installed therein. The method 700 can begin with assisting with masturbation of a male animal at operation 702. A female animal mannequin, e.g., the female animal mannequin 600 and/or 100, can be provided to the male animal at operation 704, the female animal mannequin having an orifice, e.g., the opening 536, opening inwardly therein, the orifice removably engaging with a tube, e.g., the tube 510, for receiving the insertion of an erect penis of the male animal to simulate sexual intercourse therewith. The mounting of the female animal mannequin by the male animal can be accommodated at operation 706 by positioning the orifice of the female animal mannequin to provide accessibility the male animal. The tube can be cleaned, at operation 708, after ejaculation of semen into same by the male animal. The tube can be replaced, in an optional operation 710, prior to providing the female animal mannequin to another animal. Functionality

The removable tube system 500 of the disclosed embodiments, when installed in, for example, the female animal mannequin 600, provides a solution to sexually deprived male animals by allowing for animal caretakers to conveniently replace consumable materials and/or components, e.g., the tube 510, subsequent to ejaculation there-into by the male animal. Accordingly, the masturbatory needs of the sexually deprived male animals are addressed.

One skilled in the art will appreciate that other configurations and/or placements of the components associated with the removable tube system may exist, as well as other configurations and/or placements of the female animal mannequin 600 and/or 100, dependent on the type of animal intended to please, or sexual position intended to replicate. The configurations and positions of the removable tube system 500, various components associated therewith, and the installation of the removable tube system 500 into the female animal mannequin 600 are shown as examples only and other configurations and positions may exist without departing from the scope and spirit of the disclosed embodiments.

### Advantages

The removable tube system 500 and components associated therewith provide a complete solution to sexually excited and/or frustrated male animals when installed in the female animal mannequin. Advantages include convenience in replacing the tube 510 when used by a male animal to ejaculate semen there-into by disengaging the interlocking features, e.g., protrusions 508 and 508′ with their corresponding openings 516 as substantially described in connections with FIGS. 5A-5D. Accordingly, the tube 510 may be cleaned prior to re-insertion and/or replaced by a fresh, e.g., unused, tube, suitable for another masturbatory session by the same excited male animal, or a different excited male animal.

Other advantages include variability in configuration and placement of the removable tube system 500 on the female animal mannequin 600 allowing for the application of the same for a variety of animals and/or breeds, depending on size, form, placement, and/or other preferences.

All references, including granted patents and patent application publications, referred herein are incorporated herein by reference in their entirety.

While the removable tube system 500 has been described in conjunction with a specific best mode, it is to be understood that many alternatives, modifications, and variations will be apparent to those skilled in the art in light of the preceding description. Accordingly, it is intended to embrace all such alternatives, modifications, and variations, which fall within the scope of the included claims. All matters set forth herein or shown in the accompanying drawings are to be interpreted in an illustrative and non-limiting sense.

1. An apparatus for assisting a male animal to masturbate, the apparatus comprising:
a tube with an end consisting of flat surfaces and a cavity, the tube further including protrusions extending perpendicular from opposite sides of the flat surfaces and in a same direction as the tube, and the flat surfaces extending from the cavity and extending past the protrusions; and and
a plate with a receptacle extending therefrom, the receptacle corresponding to the tube to receive the tube same upon insertion, the flat surfaces of the end engaging with a pair of openings formed through the plate to reversibly lock the tube against the plate.

2. The apparatus of claim 1 wherein the plate is installed in a female animal mannequin.

3. The apparatus of claim 1 wherein the tube and the receptacle are approximately equivalent in length for coupling there-between.

4. The apparatus of claim 1 wherein the protrusions are for reversible engagement with the pair of openings.

5. The apparatus of claim 4 wherein the protrusions slide along a passage extending from the pair of openings upon rotation of the tube relative to the plate.

6. The apparatus of claim 1 further comprising a female dog doll.

7. The apparatus of claim 1 wherein the tube is adjustable to accommodate various erect animal penis sizes.

8. The apparatus of claim 1 wherein the tube is replaceable by an unused tube in between multiple consecutive animal masturbatory sessions.

9. The apparatus of claim 1 wherein the tube accommodates cleaning of animal semen ejaculated therein.

10. The apparatus of claim 1 wherein the tube includes lubricant applied thereto.

11. The apparatus of claim 1 wherein the apparatus is configurable in various sexual positions to accommodate masturbation by the male animal.

12. The apparatus of claim 1 wherein the tube is 2 to 12 inches in length.

13. The apparatus of claim 1 wherein the tube is 1 to 6 inches in width.

14. A system for masturbation by a male dog, the system comprising:
a female dog doll with an orifice formed therein, the orifice resembling a vagina of a female dog extending towards a head of the female dog doll;
a baseplate installed in a rear region of the female dog doll, the baseplate having holes surrounding the orifice, each one of the holes having a conduit extending therefrom; and
a container insertable into the orifice and having a tube, the tube with an end consisting of flat surfaces and a cavity, the tube further including protrusions extending perpendicular from opposite sides of the flat surfaces and in a same direction as the tube, the flat surfaces extending from the cavity in the tube and extending past the protrusions, and the protrusions engage with the conduit of each one of the holes to removably couple the container to the baseplate.

15. The system of claim 14 wherein the container is removable from the orifice of the baseplate for cleaning of semen ejaculated therein.

16. The system of claim 14 wherein the container is machine-washable.

17. The system of claim 14 wherein the container is available in various colors and/or designs.

18. The system of claim 14 wherein disengagement of the protrusions from each conduit removes the container from the baseplate.

19. A method for assisting a male animal to masturbate, the method comprising the steps of:
providing a female animal mannequin to the male animal, the female animal mannequin having an orifice opening inwardly therein;
engaging a tubular insert with the orifice for receiving insertion of an erect penis of the male animal to simulate sexual intercourse with the female animal mannequin, the tubular insert including a tube with an end consisting of flat surfaces and a cavity, the tube further including protrusions extending perpendicular from opposite sides of the flat surfaces therefrom and in a the same direction as the tube, and the flat surfaces extending from the cavity in the tube and extending past the protrusions;
positioning the orifice of the female animal mannequin to provide accessibility to the male animal; and
cleaning the tubular insert after ejaculation of semen there-into by the male animal.

20. The method of claim 19 further comprising the step of:
replacing the tubular insert with an unused tubular insert prior to repeat masturbatory usage of the female animal mannequin.
